(19) United States
(12) Patent Application Publication
(10) Pub. No.: US 2017/0000381 A1
(43) Pub. Date: Jan. 5, 2017

(54) Magnetic localization of a medical device

(71) Applicant: ST. JUDE MEDICAL, CARDIOLOGY DIVISION, INC., St. Paul, MN (US)

(72) Inventors: John A. Hauck, Shoreview, MN (US); Anthony D. Hill, Minneapolis, MN (US)

(21) Appl. No.: 15/125,986

(22) PCT Filed: Mar. 21, 2015
(86) PCT No.: PCT/US15/21901

§ 371 Date: Sep. 14, 2016

Related U.S. Application Data
(60) Provisional application No. 61/968,679, filed on Mar. 21, 2014.

(51) Int. Cl. A61B 5/06; A61M 25/01; A61B 5/00

(52) U.S. Cl. CPC A61B 5/062; A61B 5/6856; A61M 25/0127; A61B 2034/2051

(57) ABSTRACT

A method of localizing a medical device in a magnetic localization field is disclosed. The medical device includes a distal region forming at least a partial loop and can be devoid of dedicated magnetic localization sensors. A conductive loop is defined by a conductive segment of the partial loop between a first end point (e.g., a first electrode on the distal region) and a second end point (e.g., a second electrode on the distal region) and a pathway connecting the end points through an electrically-conductive fluid (e.g., blood). A magnetically induced voltage is sensed in this conductive loop and then processed to localize the medical device within the magnetic localization field. Multiple such magnetically induced voltages from multiple such conductive loops can also be sensed and fit to a model to improve localization results.

### CROSS-REFERENCE TO RELATED APPLICATIONS

This application claims the benefit of U.S. provisional application No. 61/968,679, filed 21 Mar. 2014, which is hereby incorporated by reference as though fully set forth herein.

### BACKGROUND

The instant disclosure relates to medical devices. In particular, the instant disclosure relates to methods, apparatus, and systems for tracking a medical device in a magnetic localization field.

Catheters are used in a variety of diagnostic and therapeutic procedures, for example to diagnose and/or treat conditions such as atrial arrhythmias. For example, a catheter carrying one or more electrodes can be deployed and manipulated through a patient's vasculature and, once located at the intended site, radiofrequency (“RF”) energy can be delivered through the electrodes to ablate tissue. Alternatively, the electrodes can be used to create a map of the electrophysiological activity of the patient's heart.

Various systems are known for determining the position and orientation of a medical device in a human body, for example, for visualization and navigation purposes. One such system is known as a magnetic field-based positioning (or localization) system. This type of system generally includes one or more magnetic field generators attached to or placed near the patient bed or other component of the operating environment and one or more magnetic field detection coils coupled with a medical device. Alternatively, the field generators may be coupled with a medical device, and the detection coils may be attached to or placed near a component of the operating environment. The generators provide a controlled low-strength AC magnetic field in the area of interest (e.g., an anatomical region). The detection coils produce a respective signal indicative of one or more position and orientation (or localization) readings associated with the coils, and thus with the medical device. The localization readings are typically taken with respect to the field generators, such that the field generators serve as the de facto “origin” of the coordinate system of the magnetic field-based system.

### BRIEF SUMMARY

It is desirable to be able to track medical devices using a magnetic field-based localization system without the use of dedicated detection coils.

Disclosed herein is a method of localizing a medical device in a magnetic localization field, wherein the medical device comprises a distal region forming at least a partial loop, the method including: sensing a magnetically induced voltage in a conductive loop, wherein a portion of the conductive loop is defined by a conductive segment of the at least a partial loop between a first end point and a second end point and a remainder of the conductive loop is defined by a pathway connecting the first end point to the second end point through an electrically-conductive fluid (e.g., a bodily fluid, such as blood); and processing the sensed magnetically induced voltage to localize the medical device within the magnetic localization field. The medical device can be devoid of dedicated magnetic localization elements.

The first end point can be a first electrode on the distal region of the medical device (e.g., a most distal electrode), while the second end point can be a second electrode on the distal region of the medical device (e.g., a most proximal electrode). The conductive segment can be defined by a lead connected to the first electrode. It is also contemplated that ground for the sense amplifier can be defined by a third electrode on the distal region of the medical device, the third electrode being positioned intermediate the first electrode and the second electrode.

The sensed magnetically induced voltage can be processed by: inputting a signal from the first electrode to a first terminal of a sense amplifier; and inputting a signal from the second electrode to a second terminal of the sense amplifier.

In other aspects, the distal region of the medical device forms a multi-loop structure, such that the conductive loop includes multiple turns.

Alternatively, the medical device can also include one or more magnetic localization elements, which may be positioned proximally of the distal region. The localization of these magnetic localization elements can be used in conjunction with the sensed magnetically induced voltage to localize the medical device within the magnetic localization field.

In another embodiment, a method of localizing a medical device in a magnetic localization field, wherein the medical device comprises a distal region forming at least a partial loop, includes: sensing a plurality of magnetically induced voltages in a plurality of conductive loops, wherein, for each conducive loop of the plurality of conductive loops, a portion is defined by a conductive segment of the at least a partial loop between a first end point common to all conductive loops and a second end point unique to the respective conductive loop; and a remainder is defined by a pathway connecting the first end point to the second end point through an electrically-conductive fluid; and processing the plurality of sensed magnetically induced voltages to localize the medical device within the magnetic localization field.

The step of processing the plurality of sensed magnetically induced voltages can include: fitting the plurality of sensed magnetically induced voltages to a model; and using the model to derive a location of a centroid of the at least a partial loop.

According to certain aspects disclosed herein, the distal region includes a plurality of electrodes; the first end point is a reference electrode selected from amongst the plurality of electrodes; and the second end point of each conductive loop is a unique electrode, other than the reference electrode, selected from amongst the plurality of electrodes.

Also disclosed herein is a system for localizing a medical device in a magnetic localization field, wherein the medical device includes a distal region forming at least a partial loop, and wherein a conductive loop is defined by (a) a conductive segment of the at least a partial loop between a first end point and a second end point, and (b) a pathway connecting the first end point to the second end point through an electrically-conductive fluid, the system including: a sensing circuit including a sense amplifier configured to sense a magnetically induced voltage in the conductive loop; and a localization signal processor configured to process the sensed magnetically induced voltage to localize the medical device within the magnetic localization field. A first terminal of the sense amplifier can be configured to be coupled to the first end point and a second terminal of the sense amplifier can be configured to be coupled to the second end point.

The localization signal processor can be further configured to: process a plurality of sensed magnetically induced voltages from a plurality of conductive loops, wherein, for each conductive loop of the plurality of conductive loops, the first end point is common to all other conductive loops and the second end point is unique to the respective conductive loop; fit the plurality of sensed magnetically induced voltages to a model; use the model to derive a location of a centroid of the at least a partial loop; and localize the medical device within the magnetic localization field according to the location of the centroid of the at least a partial loop.

In addition, the medical device can include a magnetic localization element, which, in certain aspects, is positioned proximally of the distal region. The localization signal processor can be further configured to use a localization of the magnetic localization element in conjunction with the sensed magnetically induced voltage to localize the medical device within the magnetic localization field.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a diagrammatic view showing, in an embodiment, an exemplary magnetic field-based positioning system.

FIG. 2 illustrates a representative circular catheter, such as a circular mapping or ablation catheter.

FIG. 3 is a schematic illustration of a circular catheter connected to a sensing circuit as disclosed herein.

FIG. 4 is a partial cutaway view of an embodiment of a representative circular catheter including a magnetic field sensor.

FIGS. 5a through 5c are simplified depictions of a representative circular catheter to illustrate several parameters that describe the relationship of a virtual magnetic sensing coil to the catheter shaft.

### DETAILED DESCRIPTION

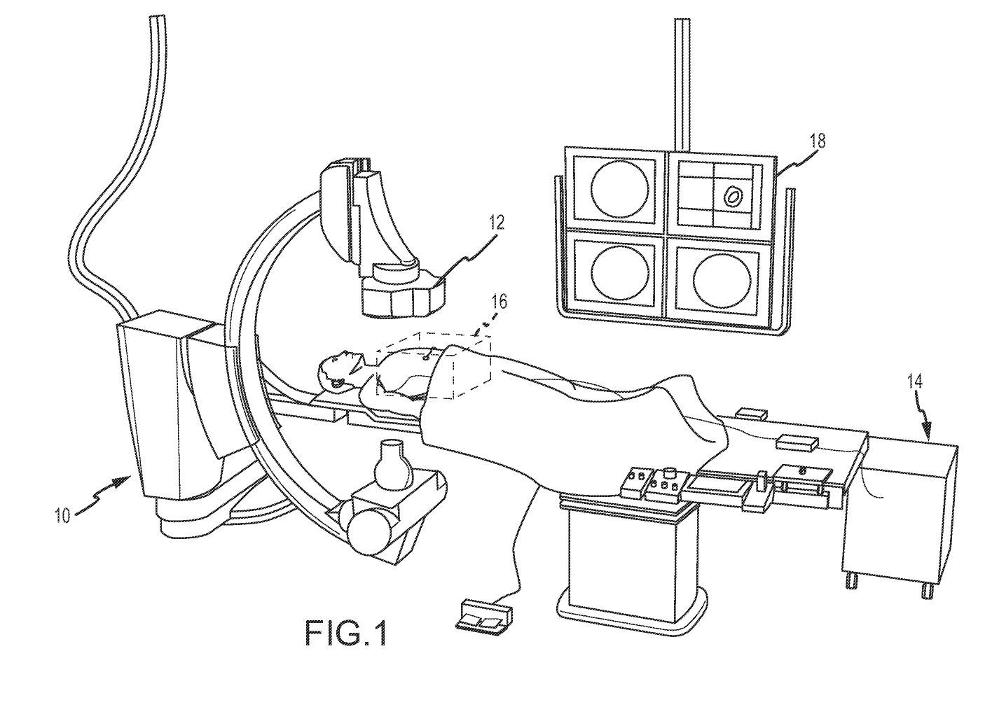

FIG. 1 is a diagrammatic view of a representative magnetic field-based localization system 10. Magnetic field-based localization system 10 can be the Mediguide™ medical guidance system of St. Jude Medical, Inc. or any other magnetic field-based localization system (e.g., the CARTO navigation and location system of Biosense Webster, Inc., the AURORA® system of Northern Digital Inc., and/or Sterotaxis' NIOBE® Magnetic Navigation System). Insofar as such systems and their operation will be generally familiar to those of ordinary skill in the art, they will be described below only to the extent necessary to understand the embodiments disclosed herein.

System 10 includes a magnetic transmitter assembly 12 and a magnetic processing core 14 for determining position and orientation readings. Magnetic transmitter assembly 12 is configured to generate a magnetic localization field in and around the patient's chest cavity as generally designated by reference numeral 16. Magnetic field sensors coupled with system 10 (e.g., carried by a medical device, such as a catheter, introduced into the patient's vasculature) are configured to sense one or more characteristics of the magnetic field and generate a respective signal that is provided to the magnetic processing core 14. The processing core 14 is responsive to these detected signals and is configured to calculate respective three-dimensional position and orientation readings for each magnetic field sensors within the magnetic localization field 16.

From an electromagnetic perspective, sensors exhibit certain common characteristics: voltage is induced on a conductive coil residing in a changing magnetic field, such as that generated by magnetic transmitter assembly 12. As the person of ordinary skill in the art will appreciate, by amplifying and processing the voltage at the magnetic field sensors, the magnitude of the potential from the magnetic field sensor attributable to each magnetic transmitter can be computed. From these values, the location and orientation of each magnetic field sensor within localization field 16 can be derived.

In particular, the potential sensed at a particular magnetic field sensor due to a magnetic field at a given frequency can be given by the following equation: V=2πNAfBμ, where N is the number of coil turns, A is loop area, f is the frequency of the coil generator current, B is magnetic field intensity along the coil axis, and μ is a gain factor if a mu-metal core is used. For example, for a sensor with 940 turns, an area of 3×10⁻⁸ m², a frequency of 5000 Hz, a magnetic field intensity of 10×10⁻⁶ T, and a gain factor of 20, the sensed voltage is about 180 μV.

In short, system 10 enables real-time tracking of each magnetic field sensor within magnetic localization field 16. The position of the sensors can be shown on a display 18 relative to, for example only, a cardiac model or geometry.

Not all medical devices, however, include dedicated magnetic field sensors. Disclosed herein are methods, systems, and apparatuses to nonetheless track such devices using magnetic field-based localization systems by defining one or more “virtual” sensor coils by reference to the device's overall geometry.

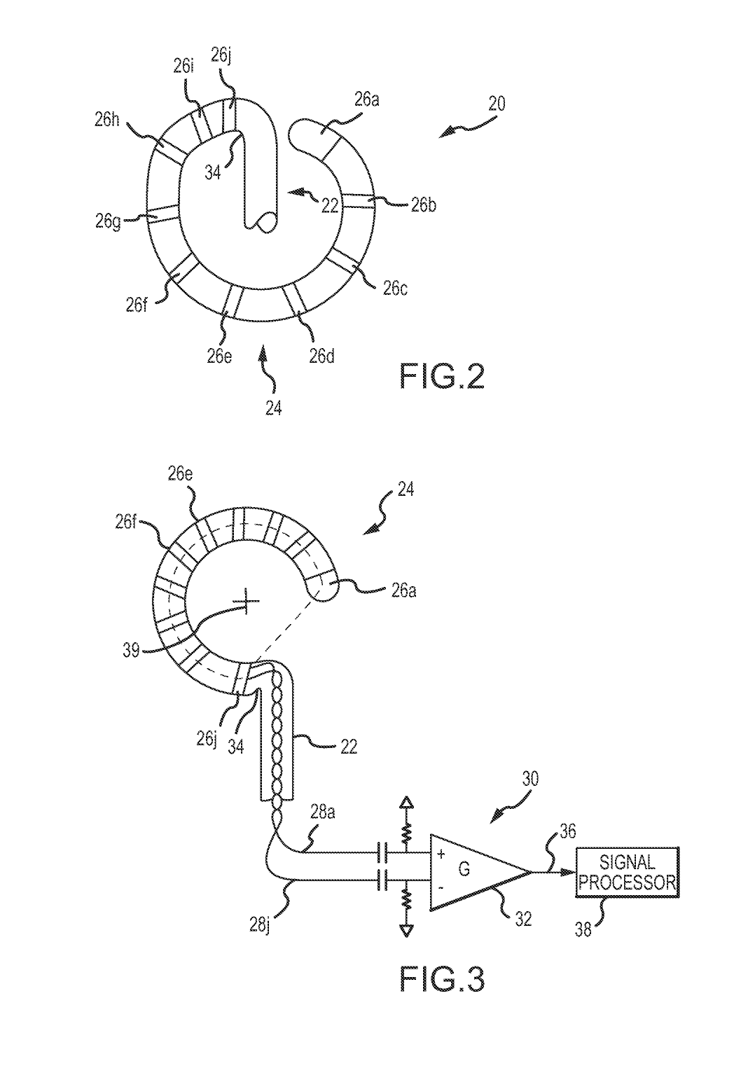

One such medical device is a circular (or “loop”) catheter 20, the general structure of which will be familiar to the ordinarily skilled artisan and a representative embodiment of which is shown in FIG. 2. As seen in FIG. 2, circular catheter 20 generally includes a shaft portion 22 including a proximal region (not shown) and a distal region 24. Distal region 24 forms at least a partial loop and can be oriented in a plane transverse to the axis of shaft portion 22, for example to enable distal region 24 to diagnose and/or treat tissue at the ostium of a pulmonary vein.

Distal region 24 further includes a plurality of electrodes 26. Electrodes 26 can be used for diagnostic purposes (e.g., to gather electrophysiology data in order to generate an electrophysiology map) and/or therapeutic purposes (e.g., to deliver ablation energy to tissue).

For purposes of illustration, FIGS. 2 and 3 depict distal region 24 as including 10 electrodes 26 (individually labeled 26a-26j for ease of reference herein). It should be understood, however, that distal region 24 can include more or fewer electrodes without departing from the scope of the present teachings.

FIG. 3 is a schematic illustration of the circular catheter 22 of FIG. 2 connected to a sensing circuit 30. As can be further seen in FIG. 3, and in some embodiments, the catheter 22 further includes a number of leads 28 extending through shaft portion 22 and connected to electrodes 26. Although only two leads (28a and 28j, corresponding to electrodes 26a and 26j, respectively) are shown in FIG. 3, this is only for the sake of clarity in the illustration. Thus, for example, each electrode 26 can be coupled to a dedicated lead 28. For purposes of explanation, however, this disclosure assumes a one-to-one relationship between electrodes 26 and leads 28 (i.e., each electrode 26 has a dedicated lead 28).

Because distal region 24 approximates a coil, it can be used to localize catheter 20 in a magnetic localization field. For example, a portion of a conductive loop can be defined by a segment of distal region 24 extending between a first endpoint (e.g., most distal electrode 26a) and a second endpoint (e.g., most proximal electrode 26j). This segment can be conductive because of electrodes a and j, as well as their respective leads 28. The remainder of the conductive loop can be defined by a pathway connecting the two endpoints through an electrically-conductive fluid. For example, when catheter 20 is placed within a patient, both endpoints (e.g., electrodes 26a and 26j) will be immersed in blood, thereby completing the conductive loop.

That is, a “virtual” magnetic sensing coil (shown in dashed lines in FIG. 3), having a single turn, can be defined by electrodes 26a and 26j and the intervening blood pool between the electrodes 26a and 26j. Using the equation given above, where N=1 and assuming that the diameter of the loop is 15 mm, f=5000 Hz, B=10×10⁻⁶ T, and a gain factor of 1, V is about 55 μV. This is a sufficiently large potential to allow for further processing and the localization of catheter 20 within magnetic localization field 16.

To this end, leads 28a and 28j for electrodes 26a and 26j can be input to a sensing circuit 30, and more particular to a sense amplifier 32. According to certain aspects, leads 28a and 28j are twisted to cancel out electromagnetic interference from external sources from a point at which the partial loop of distal region 24 begins (e.g., point 34) to the input of sensing circuit 30 (that is, through shaft portion 22 of catheter 20).

Sensing circuit 30 and sense amplifier 32 can be dedicated to magnetic localization, and can be in addition to (and in parallel to) sensing circuits and/or sense amplifiers used for the collection of electrophysiology signals via electrodes 26. To this end, it is desirable for sense amplifier 32 to be optimized for the frequency range to be sensed (e.g., in the low kilohertz range, such as between about 3 kHz and about 20 kHz). Suitable filters can also be used to reject galvanic half-cell potentials and cardiac bandwidth signals (e.g., signals below about 1000 Hz). Sense amplifier 32 should also be low noise, such as less than about 10 nV/sqrt (Hz), and provide sufficient gain (e.g., 1000 or more) to allow for subsequent signal processing and analog-to-digital conversion. The output 36 of sense amplifier 32 can be further processed (e.g., using signal processor 38, which can be part of magnetic processing core 14) to complete the localization of catheter 30.

In another aspect, sense amplifier 32 can be isolated by using an intermediate electrode (e.g., electrode 26e or 26f) as ground. This configuration advantageously minimizes the common mode potential that the endpoint electrodes (e.g., electrodes 26a and 26j) would see.

In still another aspect of the invention, multiple magnetically-induced potentials are sensed using a plurality of conductive loops. As discussed above, a portion of each conductive loop is defined by a segment of distal region 24 of catheter 20 between two endpoints (e.g., two electrodes 26) and the remainder of each conductive loop is defined by a pathway connecting the two endpoints through an electrically-conductive fluid (e.g., blood).

It is contemplated that one of the endpoints will be common to all conductive loops, while the other will be unique to a given conductive loop being sensed. For example, electrode 26e can be selected as the common endpoint, and the remaining electrodes can be used to define a total of nine conductive loops (e.g., one from endpoints 26a and 26e, one from endpoints 26b and 26e, one from endpoints 26c and 26e, and so forth). The plurality of sensed induced potentials can then be signal processed to localize the medical device, for example by fitting the plurality of sensed induced potentials to a model and using the model to derive a centroid 39 of distal region 24.

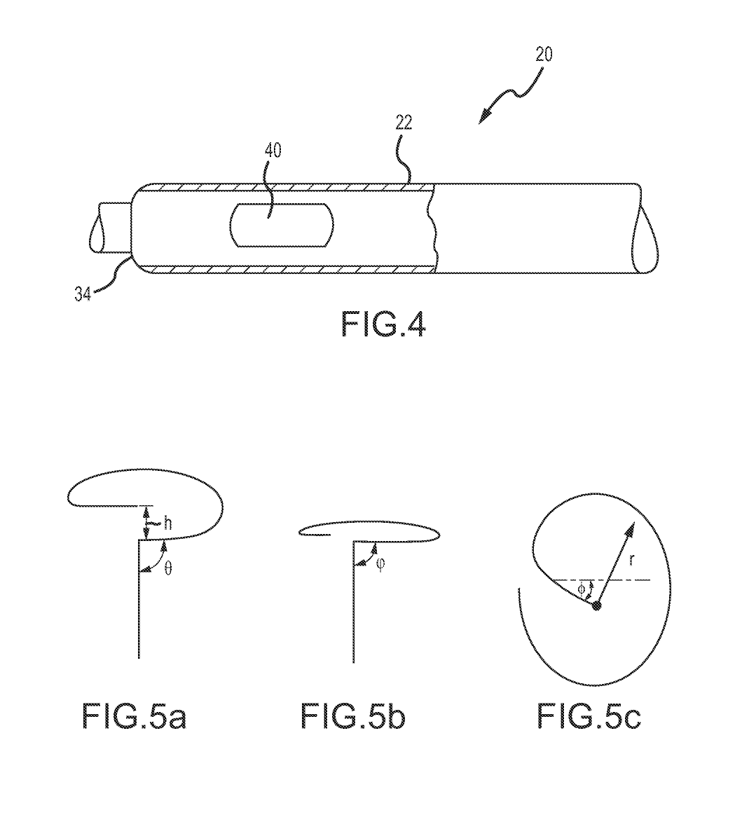

In some embodiments, one or more traditional magnetic field sensors can be used in conjunction with the virtual magnetic sensing coil described above. The inclusion of a traditional magnetic field sensor can refine the ability to track the device by compensating for variations in the impedance of the virtual magnetic sensing coil that can occur, for example, due to variations in the distance between the endpoints (e.g., electrodes 26a and 26e) and/or contact between the device and nearby tissue. FIG. 4 depicts another embodiment of catheter 20 that includes a traditional magnetic field sensor 40. In particular, traditional magnetic field sensor 40 is positioned within shaft 22, a portion of which has been cutaway to depict its interior, proximally of distal region 24 (which is not shown in FIG. 4 for clarity of illustration).

The shape of distal region 24 relative to shaft 22 can also be described with a small number of parameters identified in FIGS. 5a-5c. For the sake of illustrating these parameters in two dimensions, a simplified line representation of catheter is used throughout FIGS. 5a-5c.

FIGS. 5a and 5b are “front” and “side” views of catheter 20 (i.e., one view represents a 90 degree rotation of catheter 20 about its longitudinal axis from the other). FIG. 5a shows an angle θ, while FIG. 5b shows an angle φ, both of which describe the angle that the partial loop of distal region 24 makes relative to shaft 22. FIG. 5a also shows height h, which describes the height of the partial loop of distal region 24 if it is not in a single plane.

FIG. 5c is a view of catheter 20 looking proximally along the longitudinal axis thereof. FIG. 5c depicts the radius of curvature of the partial loop of distal region 24, denoted r, and an angle φ.

Together, traditional magnetic sensor 40 and the virtual magnetic sensing coil can be regarded as a constrained system of magnetic sensing coils. Processing core 14 can utilize the magnetic field measurements made by both sensor 40 and the virtual coil and determine values for the parameters r, h, θ, φ, and φ (e.g., best fit values to the measurements). This can allow processing core 14 to compensate for the varying impedance of the virtual sensing coil.

For example, an algorithm to localize catheter 20 can use a parameterized model of magnetic sensor 40 (e.g., position and orientation of sensor 40). The parameterized model of sensor 40 can, in turn, be used to predict a corresponding set of values for the parameters r, h, θ, φ, and φ, for example by minimizing the squared residual between predicted and actual values therefor.

Compensation for the varying impedance of the virtual sensing coil can also take into consideration the relative attenuation due to increased resistance, such that minimizing the squared residual between actual and modeled values for the parameters r, h, θ, φ, and φ will yield a best fit position and orientation for sensor 40, as well as for the shape and attenuation of the virtual sensing coil loop shape.

As the person of ordinary skill in the art will appreciate from the foregoing disclosure, if additional magnetic sensors 40 are included, there are additional measurements within what remains a single set of parameters describing catheter 20.

Although several embodiments of this invention have been described above with a certain degree of particularity, those skilled in the art could make numerous alterations to the disclosed embodiments without departing from the spirit or scope of this invention.

For example, although the description above relates to a catheter having a single loop, the ordinarily skilled artisan will understand from the foregoing disclosure how to extend the teachings herein to multi-loop (e.g., spiral) catheters, such as the Reflexion™ spiral catheter of St. Jude Medical, Inc.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

1. A method of localizing a medical device in a magnetic localization field, wherein the medical device comprises a distal region forming a portion of a conductive loop, the method comprising:
sensing a magnetically induced voltage in the conductive loop, wherein the portion of the conductive loop is defined by a conductive segment of the distal region between a first end point and a second end point and a remainder of the conductive loop is defined by a pathway connecting the first end point to the second end point through an electrically-conductive fluid; and
processing the sensed magnetically induced voltage to localize the medical device within the magnetic localization field.

2. The method according to claim 1, wherein the first end point comprises a first electrode on the distal region of the medical device and the second end point comprises a second electrode on the distal region of the medical device.

3. The method according to claim 2, wherein the conductive segment is defined by a lead connected to the first electrode.

4. The method according to claim 2, wherein the first electrode comprises a most distal electrode on the distal region of the medical device and the second electrode comprises a most proximal electrode on the distal region of the medical device.

5. The method according to claim 2, wherein processing the sensed magnetically induced voltage comprises:
inputting a signal from the first electrode to a first terminal of a sense amplifier; and
inputting a signal from the second electrode to a second terminal of the sense amplifier.

6. The method according to claim 5, wherein ground for the sense amplifier is defined by a third electrode on the distal region of the medical device, the third electrode being positioned intermediate the first electrode and the second electrode.

7. The method according to claim 1, wherein the distal region of the medical device forms a multi-loop structure, and wherein the conductive loop comprises multiple turns.

8. The method according to claim 1, wherein the medical device is devoid of dedicated magnetic localization elements.

9. The method according to claim 1, wherein the electrically-conductive fluid comprises a bodily fluid.

10. The method according to claim 1, wherein the medical device further comprises a magnetic localization element.

11. The method according to claim 10, wherein the magnetic localization element is positioned proximally of the distal region.

12. The method according to claim 10, wherein processing the sensed magnetically induced voltage to localize the medical device within the magnetic localization field further comprises using a localization of the magnetic localization element in conjunction with the sensed magnetically induced voltage to localize the medical device within the magnetic localization field.

13. A method of localizing a medical device in a magnetic localization field, wherein the medical device comprises a distal region, the method comprising:
sensing a plurality of magnetically induced voltages in a plurality of conductive loops, wherein, for each conducive loop of the plurality of conductive loops:
a portion of the conductive loop is defined by a conductive segment of the distal region between a first end point common to all conductive loops and a second end point unique to the respective conductive loop; and
a remainder of the conductive loop is defined by a pathway connecting the first end point to the second end point through an electrically-conductive fluid; and
processing the plurality of sensed magnetically induced voltages to localize the medical device within the magnetic localization field.

14. The method according to claim 13, wherein processing the plurality of sensed magnetically induced voltages comprises:
fitting the plurality of sensed magnetically induced voltages to a model; and
using the model to derive a location of a centroid of the distal region.

15. The method according to claim 13, wherein:
the distal region comprises a plurality of electrodes;
the first end point comprises a reference electrode selected from amongst the plurality of electrodes; and
the second end point of each conductive loop comprises a unique electrode, other than the reference electrode, selected from amongst the plurality of electrodes.

16. A system for localizing a medical device in a magnetic localization field, wherein the medical device comprises a distal region, and wherein a conductive loop is defined by (a) a conductive segment of the distal region between a first end point and a second end point, and (b) a pathway connecting the first end point to the second end point through an electrically-conductive fluid, the system comprising:
a sensing circuit including a sense amplifier configured to sense a magnetically induced voltage in the conductive loop; and
a localization signal processor configured to process the sensed magnetically induced voltage to localize the medical device within the magnetic localization field.

17. The system according to claim 16, wherein a first terminal of the sense amplifier is configured to be coupled to the first end point and a second terminal of the sense amplifier is configured to be coupled to the second end point.

18. The system according to claim 16, wherein the first end point comprises a first electrode on the distal region of the medical device and the second end point comprises a second electrode on the distal region of the medical device.

19. The system according to claim 18, wherein the first electrode is configured to be coupled to the first terminal of the sense amplifier via a first lead and the second electrode is configured to be coupled to the second terminal of the sense amplifier via a second lead.

20. The system according to claim 19, wherein the first lead and the second lead comprise a twisted conductor pair.

21. The system according to claim 18, wherein the first electrode comprises a most distal electrode on the distal region of the medical device and the second electrode comprises a most proximal electrode on the distal region of the medical device.

22. The system according to claim 21, wherein ground for the sense amplifier is defined by a third electrode on the distal region of the medical device, wherein the third electrode is positioned intermediate the first electrode and the second electrode.

23. The system according to claim 16, wherein the localization signal processor is further configured:
to process a plurality of sensed magnetically induced voltages from a plurality of conductive loops, wherein, for each conductive loop of the plurality of conductive loops, the first end point is common to all other conductive loops and the second end point is unique to the respective conductive loop;
to fit the plurality of sensed magnetically induced voltages to a model;
to use the model to derive a location of a centroid of the distal region; and
to localize the medical device within the magnetic localization field according to the location of the centroid of the distal region.

24. The system according to claim 16, wherein the medical device further comprises a magnetic localization element.

25. The system according to claim 24, wherein the magnetic localization element is positioned proximally of the distal region.

26. The system according to claim 24, wherein the localization signal processor is further configured to utilize a localization of the magnetic localization element in conjunction with the sensed magnetically induced voltage to localize the medical device within the magnetic localization field.
